(19) United States
(12) Patent Application Publication
(10) Pub. No.: US 2005/0014837 A1
(43) Pub. Date: Jan. 20, 2005

(54) Method for treating ocular hypertension and glaucoma

(76) Inventor: Ryuji Ueno, Montgomery, MD (US)

Correspondence Address: SUGHRUE MION, PLLC, 2100 PENNSYLVANIA AVENUE, N.W., SUITE 800, WASHINGTON, DC, 20037, US

(21) Appl. No.: 10/477,359

(22) PCT Filed: May 13, 2002
(86) PCT No.: PCT/JP02/04600

Related U.S. Application Data
(60) Provisional application No. 60/290,355, filed on May 14, 2001.

(51) Int. Cl. A61K 31/557

(52) U.S. Cl. 514/573; 514/680

(57) ABSTRACT

A method for treating ocular hypertension and glaucoma, which comprises an administration of eye drops comprising a 15-keto-prostaglandin compound as an active ingredient to a subject in need of such treatment in a single administration volume of at least 20 μL/eye is disclosed. According to the present method, the intraocular pressure reducing effect of the compound is surprisingly augmented.

### TECHNICAL FIELD

The present invention relates to a method for treating ocular hypertension and glaucoma characterized by ocular administration of eye drops comprising a 15-keto-prostagladin compound as an active ingredient in a specified volume or more.

### RELATED ART

As one method for treating ophthalmic diseases, it is a common practice to formulate pharmacologically active ingredients effective for the treatment of these diseases into eye drops, eye ointments or the like and topically apply such preparations onto a cornea, conjunctiva and the like. The administered drug, after being mixed with lacrimal fluid, mainly permeates the cornea and goes into the eyes. However, it is known that the administered drug is discharged from the conjunctival sac so speedily that a very small volume of it goes into the eyes, resulting in a very small availability of the drug within the living body.

It is generally known that increase in a single administration volume will hardly increase the pharmacological efficacy within the eyes. In the same drug concentration, the increase in the single administration volume will not increase the concentration of the drug within a precorneal tear film (PTF). For example, when 0.01% fluorescein solution is administered in different administration volumes (5, 10 and 20 μL) and their respective fluorescein concentrations in a meniscus (eyelid margin) are measured, no significant difference is observed in an apparent initial concentration among each administration volume. Further, since the initial concentration is no more than 36-45% of the concentration of the administered fluorescein, it has been revealed that the administered solution, prior to full mixture with the lacrimal fluid, is speedily discharged from the conjunctival sac. Furthermore, when 0.5% pilocarpine eye drops are administered in increased volumes (5, 10, 20 and 50 μL), miotic effects tend to increase only slightly. There is no significant difference in the effects among each administration volume (Makoto Sugaya et. al., Jpn. J. Ophthalmol. Vol.22: 127-141,1978).

Furthermore, it is reported that there was no increase in the therapeutic effect of timolol eye drop with a volume greater than 20 μL (DICP, The Annals of Pharmacotherapy, Vol.24, 1990).

Prostaglandins (hereinafter, referred to as PG(s)) are members of class of organic carboxylic acids, which are contained in tissues or organs of human or other mammals, and exhibit a wide range of physiological activity. PGs found in nature (primary PGs) generally have a prostanoic acid skeleton as shown in the formula (A):

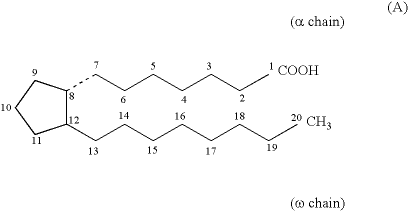

On the other hand, some of synthetic analogues of primary PGs have modified skeletons. The primary PGs are classified to PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:
Subscript 1: 13,14-unsaturated-15-OH
Subscript 2: 5,6-and 13,14-diunsaturated-15-OH
Subscript 3: 5,6-, 13,14-, and 17,18-triunsaturated-15-OH.

Further, the PGFs are classified, according to the configuration of the hydroxyl group at the 9-position, into a type (the hydroxyl group is of an α-configuration) and β type (the hydroxyl group is of a β-configuration).

PGE₁, PGE₂ and PGE₃ are known to have vasodilation, hypotension, gastric secretion decreasing, intestinal tract movement enhancement, uterine contraction, diuretic, bronchodilation and anti ulcer activities. PGF_{1α}, PGF_{2α} and PGF_{3α} have been known to have hypertension, vasoconstriction, intestinal tract movement enhancement, uterine contraction, lutein body atrophy and bronchoconstriction activities.

In addition, some 15-keto PGs (i.e. those having an oxo group at position 15 in place of the hydroxy group) and 13,14-dihydro-15-keto-PGs are known as substances naturally produced by enzymatic reactions during in vivo metabolism of primary PGs. 15-keto PG compound have been disclosed in the specification of U.S. Pat. Nos. 5,073,569, 5,166,174, 5,221,763, 5,212,324 and 5,739,161 (These cited references are herein incorporated by reference).

Moreover, it is known that some 15-keto (i.e., having oxo at the 15-position instead of hydroxy)-PGs and 13,14-dihydro-15-keto-PGs have intraocular pressure reducing effects and are effective for the treatment of ocular hypertension and glaucoma (U.S. Pat. Nos. 5,001,153; 5,151,444; 5,166,178 and 5,212,200. These publications are incorporated herein by reference).

However, it is not yet known how the 15-keto-prostaglandin compound affects the IOP reducing effects when it is administered in different volumes.

### DISCLOSURE OF INVENTION

The present inventor has conducted intensive studies on biological activities of the 15-keto-prostaglandin compound and have surprisingly found that the increase in the single administration volume will increase the IOP reducing effect and extend the retention time of the IOP reducing effect, which has resulted in the completion of the present invention.

Accordingly, the present invention relates to a method for treating a subject having ocular hypertension and glaucoma characterized by ocular administration of eye drops comprising a 15-keto-prostagladin compound as an active ingredient in a specified volume or more.

The present invention provides a method for treating ocular hypertension and glaucoma, which comprises an administration of eye drops comprising a 15-keto-prostaglandin compound as an active ingredient to a subject in need of such treatment in a single administration volume of at least 20 μL/eye.

In another aspect of the invention, the present invention provides an eye drop composition for treating ocular hypertension and glaucoma which comprises a 15-keto-prostaglandin compound as its active ingredient, which is administrated to a subject in need of such treatment in a single administration volume of at least 20 μL/eye.

In further aspect of the invention, the present invention provides use of a 15-keto-prostaglandin compound for manufacturing an eye drop composition for treating ocular hypertension and glaucoma, wherein the eye drop composition is administrated to a subject in need of such treatment in a single administration volume of at least 20 μL/eye.

### BRIEF DESCRIPTION OF THE DRAWINGS

FIG. 1. represents effect of isopropyl unoprostone eye drops on intraocular pressure (ΔIOP:mmHg) in albino rabbits (n=8)

FIG. 2. represents effect of timolol maleate eye drops on intraocular pressure (ΔIOP:mmHg) in albino rabbits (n=8)

### DETAILED DESCRIPTION OF THE INVENTION

In the present invention, the “15-keto-prostaglandin compound” (hereinafter, referred to as “15-keto-PG compound”) may include any of derivatives or analogs (including substituted derivatives) of a compound having an oxo group at 15-position of the prostanoic acid skeleton instead of the hydroxy group, irrespective of the configuration of the five-membered ring, the number of double bonds, presence or absence of a substituent, or any other modification in the α or ω chain.

The nomenclature of the 15-keto-PG compounds used herein is based on the numbering system of the prostanoic acid represented in the above formula (A).

The formula (A) shows a basic skeleton of the C-20 carbon atoms, but the 15-keto-PG compounds in the present invention are not limited to those having the same number of carbon atoms. In the formula (A), the numbering of the carbon atoms which constitute the basic skeleton of the PG compounds starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of the carboxy group (C-1). Similarly, when the number of carbon atoms is decreased in the ω-chain, the number is deleted in the order starting from position 20; and when the number of carbon atoms is increased in the ω-chain, the carbon atoms beyond position 20 are named as substituents. Stereochemistry of the compounds is the same as that of the above formula (A) unless otherwise specified.

In general, each of the terms PGD, PGE and PGF represents a PG compound having hydroxy groups at positions 9 and/or 11, but in the present specification, these terms also include those having substituents other than the hydroxy group at positions 9 and/or 11. Such compounds are referred to as 9-dehydroxy-9-substituted-PG compounds or 11-dehydroxy-11-substituted-PG compounds. A PG compound having hydrogen in place of the hydroxy group is simply named as 9- or 11-dehydroxy compound.

As stated above, the nomenclature of the 15-keto-prostaglandin compounds is based on the prostanoic acid skeleton. However, in case the compound has a similar partial construction as a prostaglandin, the abbreviation of “PG” may be used. Thus, a PG compound of which α-chain is extended by two carbon atoms, that is, having 9 carbon atoms in the α-chain is named as 2-decarboxy-2-(2-carboxyethyl)-15-keto-PG compound. Similarly, a PG compound having 11 carbon atoms in the a-chain is named as 2-decarboxy-2-(4-carboxybutyl)-15-keto-PG compound. Further, a PG compound of which ω-chain is extended by two carbon atoms, that is, having 10 carbon atoms in the ω-chain is named as 15-keto-20-ethyl-PG compound. These compounds, however, may also be named according to the IUPAC nomenclatures.

The 15-keto-PGs used in the present invention may include any PG derivatives or analogs insofar as having an oxo group at position 15 in place of the hydroxy group. Accordingly, for example, a 15-keto-PG type 1 compound having a double bond at 13-14 position, a 15-keto-PG type 2 compound having two double bond at 13-14 and 5-6 position, a 15-keto-PG type 3 compound having three double bond at 5-6, 13-14 and 17-18 position, 13,14-dihydro-15-keto-PG compound wherein the double bond at 13-14 position is single bond.

Typical examples of the compounds used in the present invention include 15-keto-PG type 1, 15-keto-PG type 2, 15-keto-PG type 3, 13,14-dihydro-15-keto-PG type 1, 13, 14-dihydro-15-keto-PG type 2, 13, 14-dihydro-15-keto-PG type 3 and the derivatives or analogs thereof.

Examples of the analogs (including substituted derivatives) or derivatives include a 15-keto-PG compound of which carboxy group at the end of α-chain is esterified; a compound of which α-chain is extended; physiologically acceptable salt thereof; a compound having a double bond at 2-3 position or a triple bond at position 5-6, a compound having substituent(s) at position 3, 5, 6, 16, 17, 18, 19 and/or 20; and a compound having lower alkyl or a hydroxy (lower) alkyl group at position 9 and/or 11 in place of the hydroxy group.

According to the present invention, preferred substituents at position 3, 17, 18 and/or 19 include alkyl having 1-4 carbon atoms, especially methyl and ethyl. Preferred substituents at position 16 include lower alkyl such as methyl and ethyl, hydroxy, halogen atoms such as chlorine and fluorine, and aryloxy such as trifluoromethylphenoxy. Preferred substituents at position 17 include lower alkyl such as methyl and ethyl, hydroxy, halogen atoms such as chlorine and fluorine, aryloxy such as trifluoromethylphenoxy. Preferred substituents at position 20 include saturated or unsaturated lower alkyl such as C1-4 alkyl, lower alkoxy such as C1-4 alkoxy, and lower alkoxy alkyl such as C1-4 alkoxy-C1-4 alkyl. Preferred substituents at position 5 include halogen atoms such as chlorine and fluorine. Preferred substituents at position 6 include an oxo group forming a carbonyl group. Stereochemistry of PGs having hydroxy, lower alkyl or hydroxy(lower)alkyl substituent at position 9 and 11 may be α, β or a mixture thereof.

Further, the above analogs may be compounds having an alkoxy, cycloalkyl, cycloalkyloxy, phenoxy or phenyl group at the end of the ω-chain where the chain is shorter than the primary PGs.

Especially preferred compounds include a 13,14-dihydro-15-keto-PG compound which has a single bond at position 13-14; a compound of which ω-chain is extended; a compound having a ring structure at the ω-chain end.

A preferred compounds used in the present invention is represented by the formula (I):

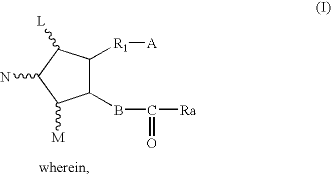

L, M and N are hydrogen atom, hydroxy, halogen atom, lower alkyl, hydroxy(lower)alkyl, or oxo, wherein at least one of L and M is a group other than hydrogen, and the five-membered ring may have at least one double bond;

A is —CH₂OH, —COCH₂OH, —COOH or a functional derivative thereof;

B is —CH₂—CH₂—, —CH═CH— or —C—C—;

R₁ is a saturated or unsaturated bivalent lower to medium aliphatic hydrocarbon residue, which is unsubstituted or substituted by halogen, alkyl, hydroxy, oxo, aryl or heterocyclic group and at least one of carbon atom in the aliphatic hydrocarbon is optionally substituted by oxygen, nitrogen or sulfur atom; and

Ra is a saturated or unsaturated lower to medium aliphatic hydrocarbon residue, which is unsubstituted or substituted by halogen atom, oxo, hydroxy, lower alkoxy, lower alkanoyloxy, cyclo (lower) alkyl, cyclo (lower) alkyloxy, aryl, aryloxy, heterocyclic group or hetrocyclic-oxy group; cyclo(lower)alkyl; cyclo(lower)alkyloxy; aryl; aryloxy; heterocyclic group; heterocyclic-oxy group.

A group of particularly preferable compounds among the above described compounds is represented by the formula (II): 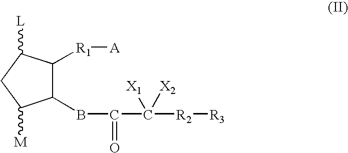
wherein L and M are hydrogen atom, hydroxy, halogen atom, lower alkyl, hydroxy(lower)alkyl or oxo, wherein at least one of L and M is a group other than hydrogen, and the five-membered ring may have at least one double bond;

A is —CH₂OH, —COCH₂OH, —COOH or a functional derivative thereof;

B is —CH₂—CH₂—, —CH═CH—, —C═—C—;

X₁ and X₂ are hydrogen, lower alkyl, or halogen;

R₁ is a saturated or unsaturated bivalent lower to medium aliphatic hydrocarbon residue, which is unsubstituted or substituted by halogen, alkyl, hydroxy, oxo, aryl or heterocyclic group and at least one of carbon atom in the aliphatic hydrocarbon is optionally substituted by oxygen, nitrogen or sulfur atom;

R₂ is a single bond or lower alkylene; and

R₃ is lower alkyl, lower alkoxy, cyclo(lower)alkyl, cyclo(lower)alkyloxy, aryl, aryloxy, heterocyclic group or heterocyclic-oxy group.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower to medium aliphatic hydrocarbon” refers to a straight or branched chain hydrocarbon group having 1 to 14 carbon atoms (for a side chain, 1 to 3 carbon atoms are preferable) and preferably 1 to 10, especially 6 to 10 carbon atoms for R₁ and 1 to 10, especially 1 to 8 carbon atoms for Rₐ.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen atom, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred example of L and M is hydroxy which has a 5-membered ring structure of, so called, PGF type.

Preferred example A is —COOH, its pharmaceutically acceptable salt, ester or amide thereof.

Preferred example B is —CH₂—CH₂—, which provide the structure of so-called, 13,14-dihydro type.

Preferred example of X₁ and X₂ is that at least one of them is halogen, more preferably, both of them are halogen, especially, fluorine that provides a structure of, so called 16,16-difluoro type.

Preferred R₁ is a hydrocarbon containing 1-10 carbon atoms, preferably, 6-10 carbon atoms. Further, at least one of carbon atom in the aliphatic hydrocarbon is optionally substituted by oxygen, nitrogen or sulfur.

Examples of R₁ include, for example, the following groups:
—CH₂—CH₂—CH₂—CH₂—CH₂—CH₂—,
—CH₂—CH═CH—CH₂—CH₂—CH₂—,
—CH₂—CH₂—CH₂—CH₂—CH═CH—,
CH₂—C≡C—CH₂—CH₂—CH₂—,
—CH₂—CH₂—CH₂—CH₂—CH (CH₃) —CH₂—
—CH₂—CH₂—CH₂—CH₂—O—CH₂—,
—CH₂—CH═CH—CH₂—O—CH₂—,
—CH₂—C≡C—CH₂—O—CH₂—,
—CH₂—CH₂—CH₂—CH₂—CH₂—CH₂—CH₂—,
—CH₂—CH═CH—CH₂—CH₂—CH₂—CH₂—,
—CH₂—CH₂—CH₂—CH₂—CH₂—CH═CH—,
—CH₂—C≡C—CH₂—CH₂—CH₂—CH₂—,
—CH₂—CH₂—CH₂—CH₂—CH₂—CH(CH₃)—CH₂—,
—CH₂—CH₂—CH₂—CH₂—CH₂—CH₂—CH₂—CH₂—,
—CH₂—CH═CH—CH₂—CH₂—CH₂—CH₂—CH₂—,
—CH₂—CH₂—CH₂—CH₂—CH₂—CH₂—CH═CH—,
—CH₂—C≡—C—CH₂—CH₂—CH₂—CH₂—CH₂—,
—CH₂—CH₂—CH₂—CH₂—CH₂—CH₂—CH(CH₃)—CH₂—

Preferred Ra is a hydrocarbon containing 1-10 carbon atoms, more preferably, 1-8 carbon atoms. Ra may have one or two side chains having one carbon atom.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

The Examples of the typical compound in the invention are 13,14-dihydro-15-keto-20-lower alkyl prostaglandin F compound and 13,14-dihydro-15-keto-17-phenyl-18,19,20-trinor-prostaglandin F compund, the derivatives or analogs thereof.

The 15-keto-PG compound of the present invention may be in the keto-hemiacetal equilibrium by formation of a hemiacetal between hydroxy at position 11 and oxo at position 15.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the 15-keto-PG compounds used in the invention include both isomers. Further, while the compounds used in the invention may be represented by a structure formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324 and 5,739,161 and U.S. patent application Ser. No. 09011218 (these cited references are herein incorporated by reference).

The term “treatment”, “treat” or “treating” used herein includes any means of control such as prevention, care, relief of the condition, attenuation of the condition and arrest of progression.

In the present invention, the term “a subject in need of such treatment” means a subject who is suffering from a disease in which a reduction in his/her intraocular pressure is desirable, for example, glaucoma and ocular hypertension, or a subject who is susceptible to suffering from such disease as discussed above. The subject may be any mammalian subject including human beings.

According to the present invention, an eye drop composition comprising the above-described 15-keto-prostaglandin compound as an active ingredient and a diluent suitable for ocular administration are prepared and administrated. The eye drop composition may be any of those manufactured according to any procedures known to the art of ophthalmic field. For example, the composition may be an ophthalmic solution or suspension that is prepared by dissolving or suspending the active ingredients in a sterile aqueous diluent such as physiological saline, buffering solution and the like. Alternatively, the composition may be that provided as a powder composition obtained by dry blending the ingredients, which is to be dissolved with an aqueous diluent suitable for ocular administration before use.

Eye drop compositions described in EP-A-0406791 (the disclosure of the publication is incorporated herein by reference) are preferred for the present invention.

If desired, additives ordinarily used in conventional eye drops may be added to the composition. Such additives may include isotonizing agents (e.g., sodium chloride), buffering agent (e.g., boric acid, sodium monohydrogenphosphate, sodium dihydrogenphosphate), preservatives (e.g., benzalkonium chloride, benzethonium chloride and chlorobutanol), thickeners (e.g., saccharide such as lactose, mannitol and maltose; e.g., hyaluronic acid or its salt such as sodium hyaluronate, potassium hyaluronate; e.g., mucopolysaccharide such as chondroitin sulfate; e.g., sodium polyacrylate, carboxyvinyl polymer and crosslinked polyacrylate).

The eye drop composition may be formulated as a sterile unit dose type product containing no preservatives.

The concentration of the active ingredient and the administration frequency of the eye drops used in the present invention may vary depending on the active ingredients used in the eye drops, the kind, such as animals or human beings, age, weight, and sex of the subject to be treated, symptoms to be treated, effects of treatment to be desired, administration methods, period of treatment and the like. Accordingly, suitable concentration and administration frequency may be chosen as desired. Taking an example of isopropyl unoprostone, which is one of preferred 15-keto prostaglandin compounds used in the present invention, eye drops containing 0.01-1.0%, preferably 0.05-0.5% of isopropyl unoprostone may be ordinarily administered to an adult human at least once a day.

As shown in the following examples, it was found in the present invention that even in the same concentration of an active ingredient, the increase in the single administration volume will increase the IOP reducing effects and also extend the retention time of the IOP reducing effects. Accordingly, in the present invention, the single administration volume per eye is at least 20 μL, preferably at least 25 μL, more preferably at least 30 μL further more preferably at least 35 μL.

According to the present invention, upper limit of the single administration volume is not particularly limited. The upper limit may be about 60 μL per eye.

The single administration volume of the eye drops may be adjusted by any conventional method, for example, by selecting suitable container or eyedropper, which can dispense the desired volume.

Accordingly, the present invention also provides an eye drop product comprising the above described composition which is incorporated in an eye drop container of which single administration volume is at least 20 μL/eye, preferably at least 25 μL/eye, more preferably at least 30 μL/eye and further more preferably at least 35 μL/eye.

When the single drop volume is less than 20 μL, which is another form of the present invention, the “single administration” may be two to three drops. In such a form, too, it is possible to obtain the same effects as in the present invention.

In the present invention, the eye drop composition may include one active ingredient only or a combination of two or more active ingredients. In a combination of plural active ingredients, their respective contents may be suitably increased or decreased in consideration of their effects, safety and the like.

Further, the eye drop composition may suitably include other pharmacologically active ingredients as far as they do not contradict the object of the present invention.

The further details of the present invention will follow with reference to the examples, which, however, are not intended to limit the present invention.

### EXAMPLE 1

Test method

0.12% isopropyl unoprostone eye drops (0.12% Rescula® eye drops) or 0.5% timolol maleate eye drops (0.5% Timoptol® eye drops) was administered once to one eye of albino rabbits (20 μL/eye or 40 μL/eye). The control group received physiological saline solution. IOP was measured with a pneumatonometer (Applanation Pneumatonograph™, Alcon Laboratories, Inc., TX, USA) before the administration and at one, two, four, six and eight hours after the administration under topical anesthesia with 0.4% oxybuprocaine hydrochloride (Benoxil® 0.4% solution, Santen Pharmaceutical Co., Ltd. Osaka, Japan)

Results

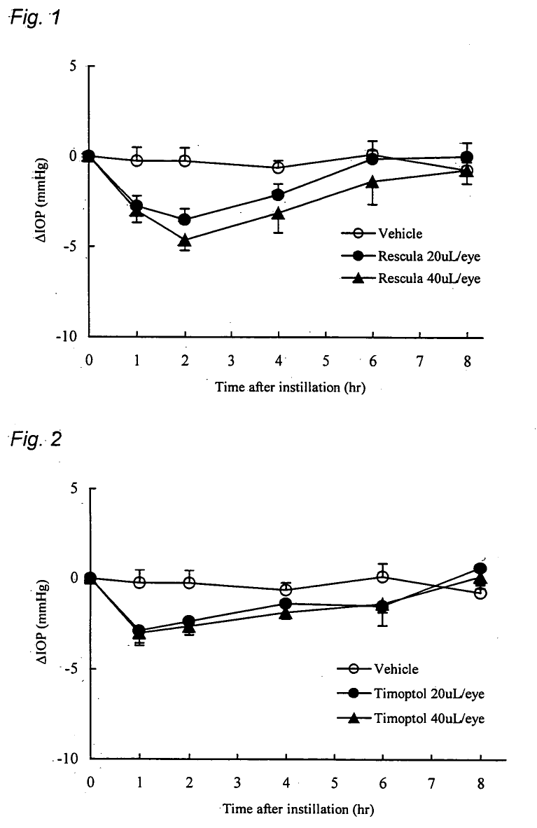

The results of IOP measurement are shown in FIGS. 1 and 2.

The administration of isopropyl unoprostone eye drops in 20 μL/eye and 40 μL/eye lowered the IOP. In both of the 20 μL/eye group and the 40 μL/eye group administered with isopropyl unoprostone eye drops, the maximum IOP reduction was seen at two hours after the administration, which were 3.5±0.6 and 4.6±0.6 mmHg, respectively. In the 20 μL/eye group, the maximum IOP reduction was seen at two hours after the administration and the IOP returned to its initial level at six hours after the administration. In the 40 μL/eye group, on the other hand, the maximum IOP reduction greater than that in the 20 μL/eye group was seen at two hours after the administration, and the IOP reducing effects was retained at six hours after the administration. From two to six hours after the administration, the IOP reduction in the 40 μL/eye group administered with isopropyl unprostone eye drops was greater than that in the 20 μL/eye group by 1.0-1.3 mmHg. The administration of isopropyl unoprostone eye drops increased the IOP reducing effects and extended the retention time of said effects depending on the administration volume.

On the other hand, the IOP reductions after the administration of both 20 μL/eye and 40 μL/eye timolol maleate eye drops were same. In both the 20 μL/eye group and the 40 μL/eye group administered with timolol maleate eye drops, the maximum IOP reduction was seen at one hour after the administration, which were 2.9±0.8 and 3.0±0.6 mmHg, respectively. The increase in the administration volume of timolol maleate eye drops neither increased the IOP reducing effects nor extended the retention time of said effects.

These results indicate that the increase in the administration volume of timolol maleate eye drops will not increase the IOP reducing effects, but the increase in the administration volume of isopropyl unoprostone eye drops will increase the IOP reducing effects and also will extend the retention time of said effects.

### EXAMPLE 2

Test method

0.12% isopropyl unoprostone eye drops (0.12% Rescula® eye drops) was administered once to one eye of albino rabbits at an administration volume of 30 μL/eye, 40 μL/eye, 50 μL/eye or 60 μL/eye. The control group received vehicle at 30 μL/eye. IOP was measured with a pneumatonometer (Applanation Pneumatonograph™, Alcon Laboratories, Inc.) before the administration and at six hours after the administration under topical anesthesia with 0.4% oxybuprocaine hydrochloride (Benoxil® 0.4% solution, Santen Pharmaceutical Co., Ltd.)

Results

The results of IOP change at six hours after the administration from pre-treatment are shown in Table 1.

The IOP reducing effects after the administration of isopropyl unoprostone eye drops at 30, 40, 50 or 60 μL/eye were increased in a volume dependent manner.

The results indicate that the increase in the administration volume of isopropyl unoprostone eye drops will increase the IOP reducing effects.

TABLE 1
Effect of isopropyl unoprostone eye drops on
intraocular pressure (ΔIOP:mm Hg) in albino rabbits

| Group | n | ΔIOP (mm Hg) |
|---|---|---|
| Vehicle 30 μL | 6 | 2.0 ± 0.7 |
| Rescula ® 30 μL | 7 | −0.9 ± 0.7 |
| Rescula ® 40 μL | 6 | −1.8 ± 1.6 |
| Rescula ® 50 μL | 6 | −2.7 ± 0.8 |
| Rescula ® 60 μL | 6 | −3.5 ± 0.8 |

(Mean ± SE)

1. A method for treating ocular hypertension and glaucoma, which comprises an administration of eye drops comprising a 15-keto-prostaglandin compound as an active ingredient to a subject in need of such treatment in a single administration volume of at least 20 μL/eye.

2. The method as described in claim 1 wherein the 15-keto-prostaglandin compound is a compound as shown by the following general formula (I). 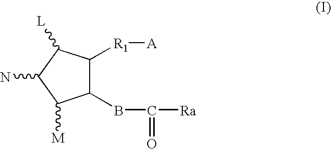
wherein L, M and N are hydrogen, hydroxy, halogen, lower alkyl, hydroxy (lower) alkyl or oxo, wherein at least one of the groups of L and M is a group other than hydrogen, and a five-membered ring may have at least one double bond;
A is —CH₂OH, —COCH₂OH, —COOH or functional derivatives thereof;
B is —CH₂—CH₂—, —CH═CH— or —C—C—;
R₁ is a saturated or unsaturated lower to medium bivalent aliphatic hydrocarbon residue, which is unsubstituted or substituted by halogen, alkyl, hydroxy, oxo, aryl or a heterocyclic group and at least one of carbon atom in the aliphatic hydrocarbon is optionally substituted by oxygen, nitrogen or sulfur atom.
Ra is a saturated or unsaturated lower to medium aliphatic hydrocarbon residue, which is unsubstituted or substituted by halogen, oxo, hydroxy, lower alkoxy, lower alkanoyloxy, cyclo (lower) alkyl, cyclo (lower) alkyloxy, aryl, aryloxy, heterocyclic group or heterocyclic-oxy group; cyclo (lower) alkyl; cyclo (lower) alkyloxy; aryl; aryloxy; heterocyclic group; heterocyclic-oxy group.

3. The method as described in claim 1 wherein the 15-keto-prostaglandin compound is a 13,14-dihydro-15-keto-prostaglandin compound.

4. The method as described in claim 1 wherein the 15-keto-prostaglandin compound is a 15-keto-20-lower alkyl-prostaglandin compound.

5. The method as described in claim 1 wherein the 15-keto-prostaglandin compound is a 13,14-dihydro-15-keto-20-lower alkyl-prostaglandin compound.

6. The method as described in claim 1 wherein the 15-keto-prostaglandin compound is a 15-keto-20-ethyl-prostaglandin compound.

7. The method as described in claim 1 wherein the 15-keto-prostaglandin compound is a 13,14-dihydro-15-keto-20-ethyl-prostaglandin compound.

8. The method as described in claim 1 wherein the 15-keto-prostaglandin compound is a 15-keto-prostaglandin F compound.

9. The method as described in claim 1 wherein the 15-keto-prostaglandin compound is a 13,14-dihydro-15-keto-20-ethyl-prostaglandin F_{2α}.

10. The method as described in claim 1 wherein the 15-keto-prostaglandin compound is a 13,14-dihydro-15-keto-20-ethyl-prostaglandin F_{2α} isopropyl ester.

11. The method as described in claim 1, wherein the single administration volume is at least 25 μL/eye.

12. The method as described in claim 1, wherein the single administration volume is at least 30 μL/eye.

13. An eye drop composition for treating ocular hypertension and glaucoma comprising a 15-keto-prostaglandin compound as an active ingredient, which is administrated to a subject in need of such treatment in a single administration volume of at least 20 μL/eye.

14. The composition as described in claim 13 wherein the 15-keto-prostaglandin compound is a compound as shown by the following general formula (I). 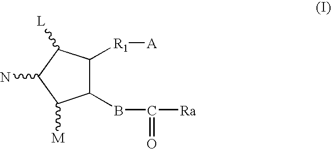
wherein L, M and N are hydrogen, hydroxy, halogen, lower alkyl, hydroxy (lower) alkyl or oxo, wherein at least one of the groups of L and M is a group other than hydrogen, and a five-membered ring may have at least one double bond;
A is —CH₂OH, —COCH₂OH, —COOH or functional derivatives thereof;
B is —CH₂—CH₂—, —CH═CH— or —C≡C—;
R₁ is a saturated or unsaturated lower to medium bivalent aliphatic hydrocarbon residue, which is unsubstituted or substituted by halogen, alkyl, hydroxy, oxo, aryl or a heterocyclic group and at least one of carbon atom in the aliphatic hydrocarbon is optionally substituted by oxygen, nitrogen or sulfur atom.
Ra is a saturated or unsaturated lower to medium aliphatic hydrocarbon residue, which is unsubstituted or substituted by halogen, oxo, hydroxy, lower alkoxy, lower alkanoyloxy, cyclo (lower) alkyl, cyclo (lower) alkyloxy, aryl, aryloxy, heterocyclic group or heterocyclic-oxy group; cyclo (lower) alkyl; cyclo (lower) alkyloxy; aryl; aryloxy; heterocyclic group; heterocyclic-oxy group.

15. The composition as described in claim 13 wherein the 15-keto-prostaglandin compound is a 13,14-dihydro-15-keto-prostaglandin compound.

16. The composition as described in claim 13 wherein the 15-keto-prostaglandin compound is a 15-keto-20-lower alkyl-prostaglandin compound.

17. The composition as described in claim 13 wherein the 15-keto-prostaglandin compound is a 13,14-dihydro-15-keto-20-lower alkyl-prostaglandin compound.

18. The composition as described in claim 13 wherein the 15-keto-prostaglandin compound is a 15-keto-20-ethyl-prostaglandin compound.

19. The composition as described in claim 13 wherein the 15-keto-prostaglandin compound is a 13,14-dihydro-15-keto-20-ethyl-prostaglandin compound.

20. The composition as described in claim 13 wherein the 15-keto-prostaglandin compound is a 15-keto-prostaglandin F compound.

21. The composition as described in claim 13 wherein the 15-keto-prostaglandin compound is a 13,14-dihydro-15-keto-20-ethyl-prostaglandin F_{2α}.

22. The composition as described in claim 13 wherein the 15-keto-prostaglandin compound is a 13,14-dihydro-15-keto-20-ethyl-prostaglandin F_{2α} isopropyl ester.

23. The composition as described in claim 13, wherein the single administration volume is at least 25 μL/eye.

24. The composition as described in claim 13, wherein the single administration volume is at least 30 μL/eye.

25. An eye drop product comprising the composition as described in any of claims 13-24, wherein the composition is incorporated in an eye drop container of which single administration volume is at least 20 μL/eye.

26. Use of a 15-keto-prostaglandin compound for manufacturing an eye drop composition for treating ocular hypertension and glaucoma, wherein the eye drop composition is administrated to a subject in need of such treatment in a single administration volume of at least 20 μL/eye.

27. Use as described in claim 26 wherein the 15-keto-prostaglandin compound is a compound as shown by the following general formula (I). 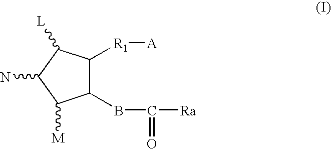
wherein L, M and N are hydrogen, hydroxy, halogen, lower alkyl, hydroxy (lower) alkyl or oxo, wherein at least one of the groups of L and M is a group other than hydrogen, and a five-membered ring may have at least one double bond;
A is —CH₂OH, —COCH₂OH, —COOH or functional derivatives thereof;
B is —CH₂—CH₂—, —CH═CH— or —C≡C—;
R₁ is a saturated or unsaturated lower to medium bivalent aliphatic hydrocarbon residue, which is unsubstituted or substituted by halogen, alkyl, hydroxy, oxo, aryl or a heterocyclic group and at least one of carbon atom in the aliphatic hydrocarbon is optionally substituted by oxygen, nitrogen or sulfur atom.
Ra is a saturated or unsaturated lower to medium aliphatic hydrocarbon residue, which is unsubstituted or substituted by halogen, oxo, hydroxy, lower alkoxy, lower alkanoyloxy, cyclo (lower) alkyl, cyclo (lower) alkyloxy, aryl, aryloxy, heterocyclic group or heterocyclic-oxy group; cyclo (lower) alkyl; cyclo (lower) alkyloxy; aryl; aryloxy; heterocyclic group; heterocyclic-oxy group.

28. Use as described in claim 26 wherein the 15-keto-prostaglandin compound is a 13,14-dihydro-15-keto-prostaglandin compound.

29. Use as described in claim 26 wherein the 15-keto-prostaglandin compound is a 15-keto-20-lower alkyl-prostaglandin compound.

30. Use as described in claim 26 wherein the 15-keto-prostaglandin compound is a 13,14-dihydro-15-keto-20-lower alkyl-prostaglandin compound.

31. Use as described in claim 26 wherein the 15-keto-prostaglandin compound is a 15-keto-20-ethyl-prostaglandin compound.

32. Use as described in claim 26 wherein the 15-keto-prostaglandin compound is a 13,14-dihydro-15-keto-20-ethyl-prostaglandin compound.

33. Use as described in claim 26 wherein the 15-keto-prostaglandin compound is a 15-keto-prostaglandin F compound.

34. Use as described in claim 26 wherein the 15-keto-prostaglandin compound is a 13,14-dihydro-15-keto-20-ethyl-prostaglandin F_{2α}.

35. Use as described in claim 26 wherein the 15-keto-prostaglandin compound is a 13,14-dihydro-15-keto-20-ethyl-prostaglandin F_{2α} isopropyl ester.

36. Use as described in claim 26, wherein the single administration volume is at least 25 μL/eye.

37. Use as described in claim 26, wherein the single administration volume is at least 30 μL/eye.

38. Use as described in any one of claims 26-37, wherein the composition is provided as an eye drop product incorporated in an eye drop container of which single administration volume is at least 20 μL/eye.
